(19) United States
(12) Patent Application Publication
(10) Pub. No.: US 2025/0144397 A1
(43) Pub. Date: May 8, 2025

(54) METHOD OF MANUFACTURING ELECTRICAL CONDUCTOR TRACKS IN A REGION OF AN INTRAVASCULAR BLOOD PUMP

(71) Applicant: Kardion GmbH, Stuttgart (DE)

(72) Inventors: Julian Kassel, Böblingen (DE); Thomas Alexander Schlebusch, Renningen (DE)

(21) Appl. No.: 19/018,309

(22) Filed: Jan. 13, 2025

Related U.S. Application Data
(62) Division of application No. 17/057,044, filed on Jun. 1, 2021, now Pat. No. 12,194,287.

(30) Foreign Application Priority Data
May 30, 2018 (DE) 102018208538.2

(51) Int. Cl. A61M 60/135; A61M 60/174; A61M 60/237; A61M 60/411; A61M 60/554; A61M 60/857

(52) U.S. Cl. CPC A61M 60/135; A61M 60/174; A61M 60/237; A61M 60/411; A61M 60/554; A61M 60/857; A61M 2205/0238; A61M 2205/3331; A61M 2205/3368; A61M 2207/00

(57) ABSTRACT

An intravascular blood pump (100) comprises a tip (110), a first region (120) with at least one blood through-opening (121), a flow cannula (130), a second region (140) with at least one blood through-opening (141), a motor-operated pump mechanism (150) and a conducting cable (170). At least in the region of the flow cannula (130), at least one electrical conductor track is realized by a surface coating structure.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is a division of U.S. patent application Ser. No. 17/057,044, entitled METHOD OF MANUFACTURING ELECTRICAL CONDUCTOR TRACKS IN A REGION OF AN INTRAVASCULAR BLOOD PUMP, and filed Jun. 1, 2021, which claims the benefit of priority to international application number PCT/EP2019/064154, entitled INTRAVASCULAR BLOOD PUMP AND METHOD FOR PRODUCING ELECTRICAL CONDUCTOR TRACKS, and filed May 30, 2019, which claims the benefit of priority to German patent application number 102018208538.2, entitled INTRAVASCULAR BLOOD PUMP AND PROCESS FOR THE PRODUCTION OF ELECTRICAL CONDUCTORS, and filed May 30, 2018, the disclosures of which are hereby incorporated by reference herein in their entirety for all purposes and forms a part of this specification.

### BACKGROUND

### Field

The present invention relates to an intravascular blood pump, which can in particular be used as a cardiac support system. The invention further relates to a method for producing electrical conductor tracks in such a blood pump.

### Description of the Related Art

So-called left ventricular assist devices (LVAD) are a known option for supporting the pumping function of the heart. These are surgically implantable mechanical pumps that support the heart. By continuously pumping blood, the blood is pumped from the left ventricle into the aorta, so that enough oxygen-rich blood can circulate in the body in a heart failure patient. So-called balloon pumps are known for this purpose. Moreover, rotary blood pumps have already been developed that can in particular also be inserted into the left ventricle and the aorta in a minimally invasive manner. The right side of the heart, for example, can also be supported in a corresponding manner. Such systems place high demands on overall size. The necessary small dimensions are achieved, for example, by reducing the wall thicknesses to a minimum. However, the integration of active electronic components or sensors in general with suitable connections is difficult. The international patent application WO 2013/160443 A1 describes an intravascular rotary blood pump in which an optical pressure sensor is integrated into the system, wherein the optical connection via optical fibers is implemented in a complex manner using neutral fibers along the flow cannula of the blood pump.

### SUMMARY

The object of the invention is to provide an improved intravascular blood pump and a method for producing such a blood pump. One object of the invention is in particular to create an intravascular blood pump, which [enables] the operation of electrical components, in particular the operation of sensors disposed, for example, in the region of a tip of the blood pump, and/or evaluation electronics disposed in said location.

This object is achieved by an intravascular blood pump having the features described herein. Such a blood pump can be produced with the method described herein.

The invention provides an intravascular blood pump, which is in particular based on the rotary pump principle, that can in particular be used as a cardiac support system. The blood pump comprises a tip, a first region with at least one blood through-opening, a flow cannula, a second region with at least one blood through-opening, a motor-operated pump device and a conducting cable for the electrical supply and control of the system. The blood pump is characterized in that at least one electrical conductor track is provided by a surface coating structure at least in the region of the flow cannula. Electrical connections and/or sensors can be realized via the electrical conductor track(s). At least one electronic component can thus be disposed in the region of the tip, in particular one or more active electronic components, for the electrical connection of which the at least one electrical conductor track is used. Such electrical conductor tracks make it possible to reduce the thickness of the electrical connecting lines to a minimum in a particularly advantageous manner. This satisfies the need for small size for such systems. Such surface coating structures in particular make it possible to bridge the region of the flow cannula. However, other regions of the blood pump can also be bridged; for example the regions of the blood through-openings and the pump device or parts thereof. Electronic components in the tip of the system can thus be electrically connected to further away regions of the system, in particular to the conducting cable, so that power transmission and/or data transmission from or to external control devices and/or evaluation devices, for example, is possible. The invention permits a very advantageous electrical contacting of electronic components in the tip or also at another position, whereby the implementation of the electrical contacting or connection to the conducting cable can be very thin and space-saving and, at the same time, very firm, stable and reliable due to the electrical surface functionalization. The assembly process required for this can be realized in a cost-effective manner.

The electronic components in the region of the tip can in particular be sensors, for example pressure sensors, flow measuring sensors, temperature sensors, etc. Optical sensors, acceleration or rotation rate sensors and acoustic sensors (microphones), for example, are possible as well. Any sensors or other electronic components and electrode surfaces that are suitable for medical monitoring of the patient and/or the function of the intravascular blood pump and/or for controlling the blood pump can be used.

As an alternative or in addition to an electrical connection of electronic components via the conductor tracks, sensors can be realized using the conductor tracks themselves, for example strain sensors and/or breakage sensors and/or temperature sensors. In this way, sensors can be integrated into the surface structure in a very advantageous manner. The use of exposed electrodes for recording electrical excitation signals or for performing an electrical impedance measurement is possible as well. Such sensors can be realized by sensor regions within the surface coating structure which comprise meandering conductor tracks. The conductor tracks in the sensor region(s) can also be made of a different material than the conductor tracks outside the sensor regions. The conductor tracks in a sensor region can be made of platinum, for example, which allows the sensor region to be used as a temperature sensor. Such sensor regions can furthermore also be used as electrical sensors, so that the sensors can be used for dielectric characterization of the surrounding blood, for example. The coupling can be conductive or capacitive, comparable to an impedance spectroscopy. It is also possible to integrate a thin surface wave sensor, for example as a thin ceramic disc, for example for determining the blood viscosity.

The flow cannula of the intravascular blood pump preferably comprises one or more coatable materials. A hose guide made of a coatable material can in particular be provided. The surface coating structure is applied to the coatable material or materials to realize the electrical conductor tracks. As a general rule, it is useful for the flow cannula to be flexible. For this purpose, the hose guide can, for example, be equipped with a flexible skeletal structure, for example a spiral structure. Other options include zigzag or wave structures. The flexible structure (e.g. the spiral structure) is expediently designed such that there is a continuous web structure on which the electrical conductor tracks are held. Such flexible structures are particularly advantageously at least partially made of the coatable material. Metallic materials, for example titanium and/or stainless steel, can be used as coatable materials. Nickel-titanium alloys (NiTiNol), which are already used in medical technology due to their particularly advantageous properties, are particularly preferred. In addition to their advantages in terms of their deformation properties, nickel-titanium alloys also have the advantage of being directly coatable. Other suitable coatable materials are, for example, glass and/or ceramic.

The surface coating structure can preferably have a multilayer structure, for example a two-layer structure, whereby the lower layer in the space between two conductor structures can be used for metallizing a further conductor layer, so that multiple conductor track layers are nested inside one another. On the one hand, this allows the overall conductor width to be reduced. On the other hand, the layer thickness of the conductor structure as a whole is reduced.

For electrical contacting of the conductor tracks it is preferable that electrical contact pads are provided. The contact pads can be disposed at the end of the flow cannula, for example, opposite the tip of the system.

The invention further involves a method for producing electrical conductor tracks at least in the region of a flow cannula of an intravascular blood pump, wherein, concerning this blood pump, reference is made to the above description. The electrical conductor tracks are produced using a surface coating, in particular using surface lithographic techniques. First and foremost, optical lithographic methods (e.g. UV lithography) can be used. Flat 2D wafer processes can be used on cylindrical bodies, for example, so that conventional lithography processes can in principle be used by adapting the exposure devices. Photolithographic methods, in particular three-dimensional UV photolithographic methods, are particularly suitable. Magnetron sputtering and, if necessary, wet chemical etching methods can in particular be used to produce the surface structuring.

In a preferred embodiment of the method, after a possibly necessary initial cleaning and surface activation of the material to be coated, an insulating base layer can first be applied to the coatable material. This can be an oxide layer that is applied by sputtering, for example, or a polyimide. A photoresist is then applied and structured in accordance with the conductor tracks to be applied. For this purpose, a lithography mask is expediently applied, for example made of chrome-coated quartz substrate, before the photoresist is exposed and developed. The metallic conductor track structure is then applied by sputtering. For reasons of biocompatibility, gold is preferably used as the material for the conductor tracks. The photoresist is then removed. Finally, an electrically insulating and preferably biocompatible surface is applied. This too can again be done by sputtering oxide, for example, or by applying polyimide or parylene or something else. The layer thickness of the resulting sputtered surfaces is preferably in a range of several hundred nanometers.

In particular for applications that require a high conductivity of the conductor track structures, a conductor track structure with an increased layer thickness (for example several micrometers) can be provided using the design variant of the method described in the following. For this purpose, an in principle complete conductive surface coating is produced first. This is windowed by a structured photoresist and the exposed windows are then galvanically thickened. Specifically, in this variant, after a possibly necessary initial cleaning and surface activation, an insulating base layer is applied first, for example an oxide layer by sputtering or a base layer of polyimide. Then an initial metallic conductor layer (e.g. gold) is applied. A photoresist is applied to the initial conductor layer and structured in accordance with the conductor tracks to be applied.

The exposed metallic conductor tracks or the exposed windows are then thickened using a wet chemical electroplating process so that the desired conductivity can be produced in the exposed metal structures. The photoresist is removed. To remove the initial metal conductor layer outside the conductor track structures, the surface is etched so that the electrical conductor track structures are exposed. Lastly, an electrically insulating and preferably biocompatible surface is applied, for example by sputtering oxide or by applying polyimide or parylene or other materials.

In addition to the surface structuring for producing the conductor tracks, the process preferably also includes the structuring of the pipe material, in which a web structure is produced on which the conductor tracks are held (for example a spiral structure). This structuring can occur before or after the production of the conductor tracks. Finally, the windows of the web structure are closed with silicone or polyurethane, for example.

### BRIEF DESCRIPTION OF THE DRAWINGS

Further features and advantages of the invention emerge from the following description of design examples in conjunction with the drawing. The individual features can be realized individually or in combination with one another.

Advantageous embodiments of the invention are shown schematically in the drawings and are described in the following.

The figures show:

FIG. 1 a sectional view of a human heart and lung with an inserted intravascular blood pump;

FIG. 2 components of an intravascular blood pump (LVAD system);

FIG. 3 an isometric illustration of a flexible hose guide of the flow cannula of an intravascular blood pump;

FIG. 4 a detail view of the hose guide of a flow cannula having a surface coating structure according to the invention for the formation of conductor tracks;

FIG. 5 a detail view of the hose guide of a flow cannula having a surface coating structure according to the invention with the configuration of sensor regions by the conductor tracks;

FIG. 6 a detail view of the hose guide of a flow cannula having a surface coating structure according to the invention showing electrical contact pads;

FIG. 7 a detail view of a cross-section through a flow cannula having a surface coating structure according to the invention;

FIG. 8 a further detail view of a cross-section through a flow cannula having a surface coating structure according to the invention with a two-layer structure;

FIG. 9 a further detail view of a cross-section through a surface coating structure with a multilayer structure; and

FIG. 10 a further detail view of a cross-section through a surface coating structure with a multilayer structure and shielding.

### DETAILED DESCRIPTION

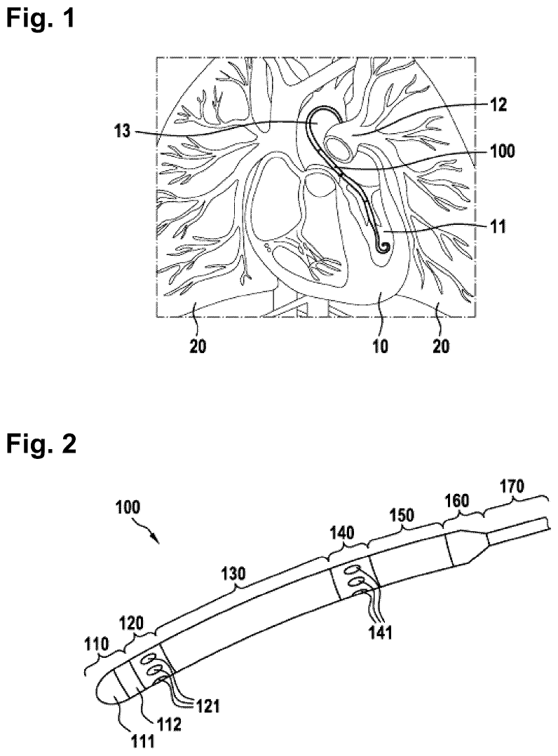

FIG. 1 shows a human heart 10 and the surrounding lungs 20, wherein an intravascular blood pump 100 is inserted in the left ventricle 11. Pumping the blood pump 100 supports the pumping function of the heart 10 by moving oxygen-rich blood coming into the left ventricle 11 from the pulmonary vein 12 into the aorta 13. The intravascular blood pump can be designed for continuous pumping, for example, or the pump is based on a pulsatile system, for example, in which the pump speed is modulated.

FIG. 2 schematically shows the components of an intravascular blood pump 100 that is equipped according to the invention with a surface coating structure for the formation of electrical conductor tracks. The blood pump 100 comprises a tip 110, wherein one or more electronic components 112, in particular sensors, can be provided in a region within the tip 110. The tip is closed by a slidable cap 111. A first region 120 (inlet cage) with blood through-openings 121 adjoins the tip 110. Blood can be drawn into the blood pump, for example from the left ventricle, through the blood through-openings 121. This is adjoined by a flow cannula 130 and a second region 140 (impeller cage) having further blood through-openings 141. This is adjoined by region 150 for a motor-operated pump device. Inside the region 140 there is a rotor (impeller), for example, that is operated via the pump device 150, so that the pumped blood can exit through the blood through-openings 141. The pump device 150 is adjoined by a back end 160, via which the electrical connection is made. A supply cable 170 is provided for electrical supply and control. The motor-operated pump device is preferably a rotary pump (flow machine), wherein a reversal of the conveying direction can also be provided if necessary.

The surface coating structure according to the invention allows sensors or sensor regions, for example breakage sensors or strain sensors or temperature sensors, to be realized, in particular in the region of the flow cannula. The surface coating structures can also be used to electrically connect any existing electronic components 112 of the tip 110 to the supply cable 170. This allows the length of the flow cannula 130 in particular, but also the regions 120 and 140 and the region with the motor-operated pump device 150, to be bridged. Different components can be combined and realized as one structural element. For example, the first region 120 can be combined with the flow cannula 130 to one structural element, which can then very advantageously be equipped with the surface coating structure according to the invention for the formation of conductor tracks.

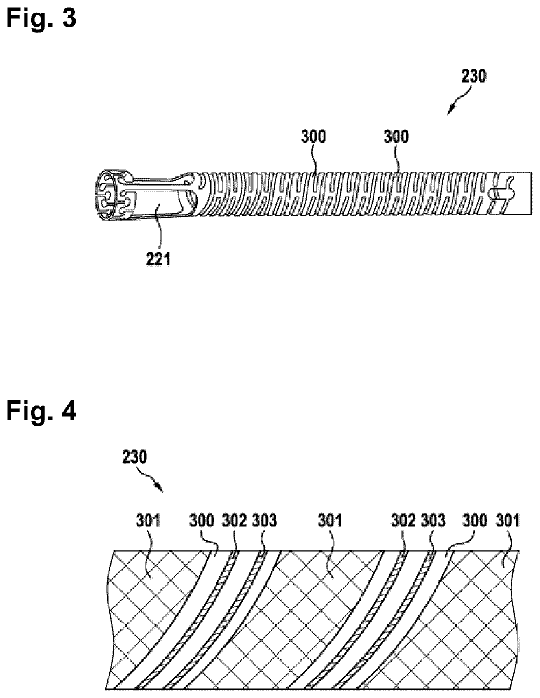

FIG. 3 shows a combined configuration of the first region with blood through-openings 221, which is directly adjoined by the flow cannula 230. The flow cannula 230 is advantageously realized as a flexible inlet hose or as a flexible hose guide. In this example, the flexible flow cannula 230 is realized by a spiral-shaped structure formed by circumferential windowed webs 300. A laser-structured tube made of NiTiNol material, for example, can be provided as the coatable material for this purpose. On the right side of the laser structured tube there is an elongated opening, which is provided for the passage of a guide wire in a per se known manner during the implantation process. The skeleton or web structures 300 of the NiTiNol material are electrically functionalized by surface coating for the formation of the conductor tracks, whereby the conductor tracks can in particular be used for electrically connecting electronic components and/or for the formation of sensors. The spiral structure of the NiTiNol tube can be produced by laser structuring. The exposed windows of the laser structured form can be closed by flexible materials, for example by silicone or polyurethane. The flexibility of the hose guide can also be achieved with other structures, for example by zigzag or wave patterns. The surface coating structure as such can be applied according to the method already described above. In this context, reference is also made to an article by Bechtold et al. (Biomed Microdevices, 2016 December; 18(6): 106) and an article by Lima de Miranda et al. (Rev. Sci. Instrum., 2009 January; 80(1): 015103), whereby these articles deal with surface structuring in general. Bechtold et al. describe the coating of thin films made of a nickel-titanium alloy to form insulated electrodes on the outer surface. Lima de Miranda et al. describe a rotational UV lithography for cylindrical geometries. The laser structuring of the NiTiNol tube to form the spiral structure, for example, can take place before or after the electrical functionalization.

FIG. 4 shows a detail view of the resulting exemplary conductor track structures on the flow cannula 230. The webs 300 of the laser-structured spiral structure (see FIG. 3), which to a certain extent form the framework of the flexible flow cannula 230, leave windows 301 open. The windows 301 are preferably closed in a flexible manner, for example using silicone or polyurethane. The webs 300 together with the closed windows 301 form the hose guide of the flow cannula 230. According to the invention, electrical conductor track structures 302, 303 are applied to the webs 300 using lithography and coating technologies.

For the actual production of the electrical conductor tracks, a lithography mask comprising the corresponding coating structures (electrical conductor track structures) is applied for each layer. The lithography mask can be a chrome-coated quartz substrate, for example. Non-conductors such as photoresist or polyimide can be applied over a large area by dipping, for example. Non-conductors such as parylene C can be deposited in a vacuum, for example. Initial metallic layers are in particular applied by sputtering, thicker layers by electrodeposition.

There are two main approaches that can be used in the production process: According to Method 1, the tube material (for example NiTiNol) is first provided with the electrical surface coating for the formation of the conductor tracks. In the next step, the flexible structure is produced, for example, by laser cutting (laser structuring), whereby the coating structure and the laser cutting contour are geometrically aligned to one another. In the last step, the windows of the flexible structure are closed, for example by dipping or overmolding. According to Method 2, the pipe material is structured first. The surface functionalization for the formation of the conductor tracks is then produced using the lithographic processes. Lastly, the windows of the flexible structure are closed as in Method 1. Method 1 has the advantage that the lithography process is simplified. Method 2 has the advantage that shape embossments in the NiTiNol material are possible directly after the structuring of the pipe material; for example to “save” bends or cross-sectional changes to the cross-section of the starting material (e.g. widenings of the cross-section). Because of the process temperatures needed for the shape embossment, it is generally advantageous to perform this step before the lithographic surface coating.

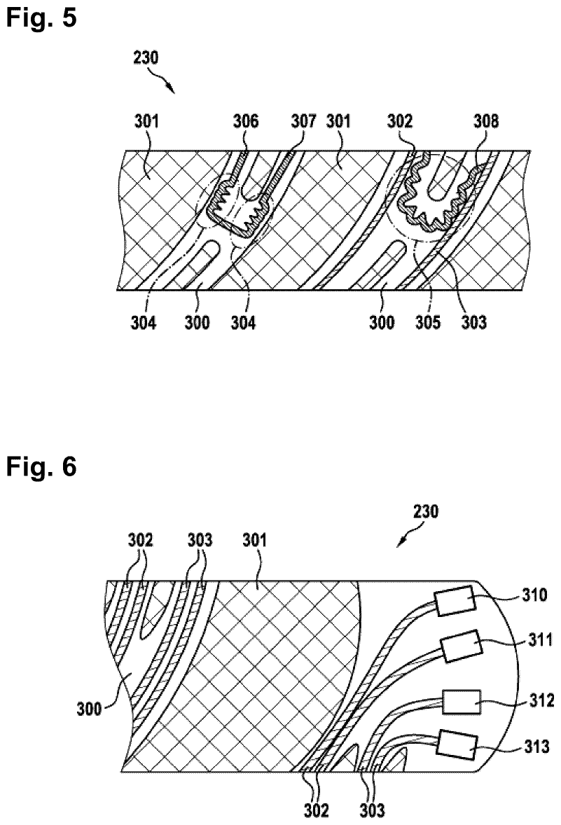

FIG. 5 shows particularly preferred configurations of the conductor tracks, in which the conductor track structure is designed as a sensor (left) or as an electrical connection and additionally as a sensor (right). As in FIG. 4, the flow cannula 230 is equipped with conductor tracks 302,303, which are formed by surface structuring of the webs 300 of the flow cannula 230 (right part of the illustration). Meandering conductor tracks are provided as well, which form the sensor regions 304 (left) or the additional sensor region 305 (right). Straight sections of the conductor tracks can be provided between individual sensor regions 304, or the sensor region 305 is formed by a continuously meandering conductor track. The input and output lines 306, 307 of the sensor regions 304 can be made of a different material than the sensor regions themselves. A plurality of sensor regions can be implemented via separate input lines or even with a common return channel line 308, for example.

For a temperature sensor, for example, it can be provided that the conductor tracks of the sensor regions 304 or 305 are made of platinum, because platinum has a very linear resistance-temperature relationship. The input and output tracks 306, 307, 308 expediently have the lowest possible resistance in order to have little influence on the sensor signal. The conductor track structures can also be used as strain or breakage sensors, for example. They can also be used as capacitive sensors, electrode surfaces or contact pads for further sensors, for example.

FIG. 6 shows a preferred electrical contacting of the conductor tracks 302, 303 via electrical contact pads 310, 311, 312, 313. This electrical contacting can take place, for example, at the end of the flow cannula 230, i.e. in the direction toward the second region 140. However, it is also possible for the conductor tracks to also be guided over other components of the blood pump, for example over the region 140, 150 to the electrical connection region 160. The electrical connection can be established by conductive gluing, soldering, bonding or frictional connection, for example. The connection can be made directly from NiTiNol component to NiTiNol component, for example, or from NiTiNol component directly to a cable or a thin-film substrate, depending on the configuration of the blood pump.

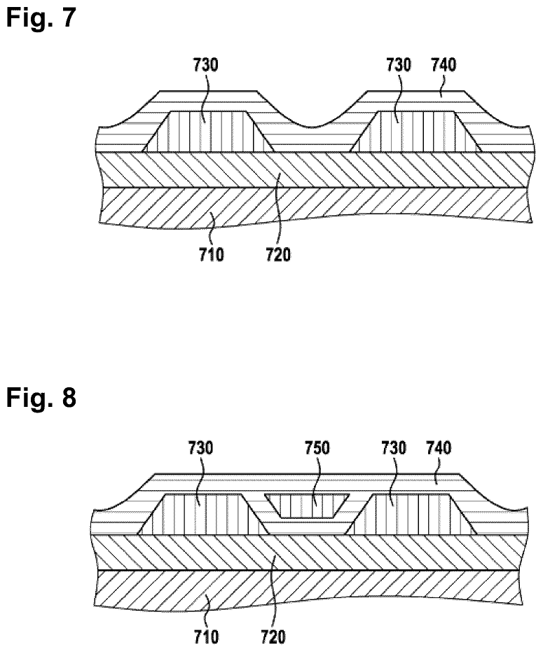

FIG. 7 shows a cross-section through the resulting layer structure that realizes the electrical conductor tracks. 710 represents the underlying NiTiNol structure or another coatable material as the support structure of the flow cannula. 720 represents an insulating base layer, for example made of silicon oxide or polyimide. 730 shows the metallic conductor track structures, for example made of gold. 740 represents an insulating cover layer, for example made of silicon oxide, polyimide or parylene. A multilayer structure, for example a two-layer structure as illustrated in FIG. 8, can be created by repeating the surface coating several times (surface lithography). 710, 720, 730 and 740 represent the coatable structure, the insulating base layer, the first layer of the conductor track structures or the insulating cover layer, as in FIG. 7. A further conductor track 750 disposed at a slightly higher level is additionally provided in the spaces between the conductor track structures 730. During production, the space (empty space) between the conductor track structures 730 on the lower layer is used for the metallization of the upper layer by disposing the metallic conductor layer in this space. This offset arrangement of the conductor tracks on different levels prevents the formation of larger protrusions or roughnesses of the surface structure in the regions in which metallic conductor tracks would be on top of one another. This can occur in particular in higher multilayer structures having six or more layers. In this respect, this embodiment with an offset arrangement has the advantage over a purely coaxial embodiment that the resulting layer thickness of the conductor structure as a whole is reduced. This embodiment is also particularly advantageous compared to a coplanar design, because the overall conductor width is reduced. If an offset arrangement of the conductor tracks is not desired or possible, it is alternatively also possible to compensate any unevenness that may occur due to superimposed conductor tracks, for example with a silicone layer or the like.

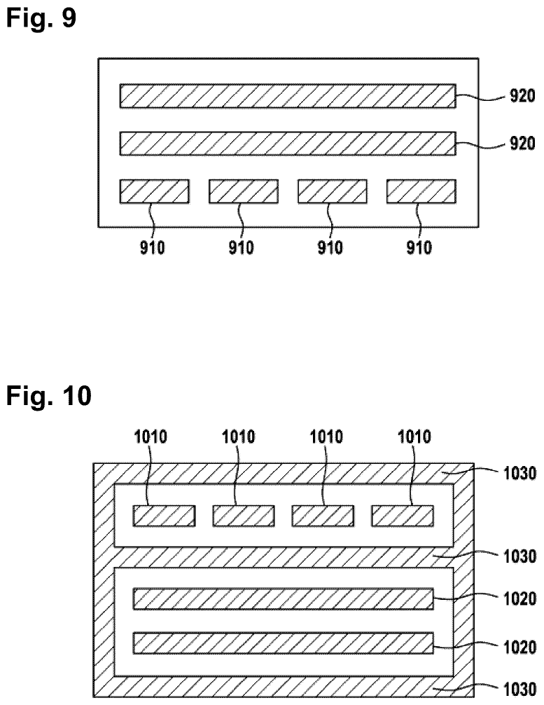

FIG. 9 shows a further structure of a multilayered conductor track structure. Four narrow conductor tracks 910 and two wide conductor tracks 920 are disposed one above the other on the coatable material (not shown in detail). The narrow conductor tracks 910 serve as a communication bus for a pressure sensor and a temperature sensor in the tip of the blood pump, for example. The wide conductor tracks 920 have a lower resistance (electrical power) and are used, for example, to connect an ultrasonic element in the tip of the blood pump. To produce such a structure, a total of seven layers are required for the surface coating. FIG. 10 shows a similar example of a 5 multilayered structure having four narrow conductor tracks 1010 and two wide conductor tracks 1020. Metallizations, which shield the conductor tracks 1010 and 1020 against one another and to the outside, are additionally provided as a shielding 1030, so that a defined line impedance and less high-frequency radiation are achieved along with a shielded routing of the signals. A total of 11 layers are required to produce such a structure. In the contact pad region, the up to 11 layers can expediently be widened accordingly and, for example, passed into the top metal layer through a vertical through-connection.

1. An intravascular blood pump comprising:
a tip;
a first region comprising at least one blood through-opening;
a flow cannula;
a second region comprising at least one blood through-opening;
a motor-operated pump device; and
a conducting cable,
wherein at least one electrical conductor track comprising a surface coating structure in at least a region of the flow cannula; and
wherein the at least one electrical conductor track extends about a spiral structure of the flow cannula.

2. The blood pump according to claim 1, further comprising at least one electronic component disposed in a region of the tip, wherein the at least one electrical conductor track provides an electrical connection for the at least one electronic component.

3. The blood pump according to claim 2, wherein the at least one electrical component comprises an ultrasonic element.

4. The blood pump according to claim 2, wherein the at least one electrical component comprises a pressure sensor.

5. The blood pump according to claim 2, wherein the at least one electrical component comprises a temperature sensor.

6. The blood pump according to claim 1, wherein one or more sensors are integrated into the surface coating structure.

7. The blood pump according to claim 6, wherein the one or more sensors comprise at least one of strain sensors, breakage sensors, and temperature sensors.

8. The blood pump according to claim 6, wherein the at least one electrical conductor track comprises one or more meandering conductor tracks forming one or more sensor regions of the one or more sensors.

9. The blood pump according to claim 8, wherein the one or more meandering conductor tracks forming the one or more sensor regions are at least partially formed of a different material than one or more conductor tracks outside the sensor regions.

10. The blood pump according to claim 8, wherein the one or more meandering conductor tracks forming the one or more sensor regions are formed of platinum.

11. The blood pump according to claim 1, wherein the flow cannula comprises a coatable material, wherein the surface coating structure is configured to be applied to the coatable material to form the electrical conductor tracks.

12. The blood pump according to claim 11, wherein the coatable material comprises at least one of: nickel-titanium alloys, titanium, stainless steel, glass, and ceramic.

13. The blood pump according to claim 1, wherein the surface coating structure comprises a multilayer structure.

14. The blood pump according to claim 1, wherein electrical connection of the conductor tracks is established by a frictional connection.

15. A method of manufacturing electrical conductor tracks in a region of an intravascular blood pump, the method comprising:
applying an insulating base layer to a coatable material;
applying a photoresist material;
applying a conductor track structure, wherein the conductor track structure is applied by sputtering;
removing the photoresist material; and
applying an electrically insulating surface, wherein the electrical insulating surface is biocompatible.

16. The method according to claim 15, wherein the intravascular blood pump comprises:
a tip;
a first region comprising at least one blood through-opening;
a flow cannula;
a second region comprising at least one blood through-opening;
a motor-operated pump device; and
a conducting cable.

17. The method according to claim 16, further comprising at least one electronic component disposed in a region of the tip, wherein at least one electrical conductor track provides an electrical connection for the at least one electronic component.

18. A method of manufacturing electrical conductor tracks in a region of an intravascular blood pump, the method comprising:
applying an insulating base layer to a coatable material;
applying an initial metallic conductor layer, wherein the initial metallic conductor layer is applied by sputtering;
applying a photoresist material;
thickening exposed portions of the initial metallic conductor layer using a wet chemical electroplating process;
removing the photoresist material;
removing portions of the initial metallic conductor layer outside conductor tracks;
applying an electrically insulating surface, wherein the electrically insulating surface is biocompatible.

19. The method according to claim 18, wherein the intravascular blood pump comprises:
a tip;
a first region comprising at least one blood through-opening;
a flow cannula;
a second region comprising at least one blood through-opening;
a motor-operated pump device; and
a conducting cable.

20. The method according to claim 19, further comprising at least one electronic component disposed in a region of the tip, wherein at least one electrical conductor track provides an electrical connection for the at least one electronic component.
